(19) United States
(12) Patent Application Publication
(10) Pub. No.: US 2010/0221805 A1
(43) Pub. Date: Sep. 2, 2010

(54) METHODS FOR PRODUCING FERMENTATION PRODUCTS

(75) Inventor: Guillermo Coward Kelly, Wake Forest, NC (US)

Correspondence Address: NOVOZYMES NORTH AMERICA, INC., 500 FIFTH AVENUE, SUITE 1600, NEW YORK, NY, 10110, US

(73) Assignee: NOVOZYMES NORTH AMERICA, INC., Franklinton, NC (US)

(21) Appl. No.: 12/663,557

(22) PCT Filed: Jun. 9, 2008
(86) PCT No.: PCT/US08/66250

§ 371 Date: Dec. 8, 2009

Related U.S. Application Data
(60) Provisional application No. 60/942,876, filed on Jun. 8, 2007.

(51) Int. Cl. C12P 7/10

(52) U.S. Cl. 435/165

(57) ABSTRACT

The invention relates to methods for producing a fermentation product from a lignocellulose-containing material comprising: i) pre-treating lignocellulose-containing material; ii) introducing pre-treated lignocellulose-containing material into medium comprising fermentable sugars derived from starch-containing material; ii) fermenting using a fermenting organism.

### TECHNICAL FIELD

The present invention relates to methods for producing fermentation products from lignocellulose-containing material.

### BACKGROUND OF THE INVENTION

Due to the limited reserves of fossil fuels and worries about emission of greenhouse gases there is an increasing focus on using renewable energy sources.

Production of fermentation products from lignocellulose-containing material is known in the art and includes pre-treating, hydrolyzing, and fermenting the lignocellulose-containing material. Unfortunately, pre-treatment results in release of compounds, e.g., phenolics and furans, which inhibit and/or inactivate the performance of enzymes and are toxic to fermentation organisms. It is possible to remove toxic compounds, e.g., by washing the pre-treated lignocellulose-containing material, but it is expensive and/or cumbersome to do so.

Consequently, there is a need for providing further methods and processes for producing fermentation products from pre-treated lignocellulose materials especially un-detoxified pre-treated lignocellulosic material.

### SUMMARY OF THE INVENTION

In the first aspect the invention relates to a method for producing a fermentation product from lignocellulose-containing material, wherein the method comprises:

i) pre-treating lignocellulose-containing material;

ii) introducing pre-treated lignocellulose-containing material into medium comprising fermentable sugars derived from starch-containing material; and

iii) fermenting using a fermenting organism.

In the second aspect the invention relates to a process for producing a fermentation product from a combination of starch-containing material and lignocellulose-containing material comprising the steps of:

a) liquefying starch-containing material;

b) saccharifying; and

c) fermenting using a fermenting organism;

wherein pre-treated lignocellulose-containing material is added before and/or during fermentation.

In the final aspect the invention relates to a process for producing a fermentation product from a combination of starch-containing material and lignocellulose-containing material comprising the steps of:

i) saccharifying the starch-containing material at a temperature below the initial gelatinization temperature:

ii) fermenting using a fermenting organism;

wherein pre-treated lignocellulose containing material is added before and/or during fermentation.

### BRIEF DESCRIPTION OF THE FIGURES

FIG. 1 shows the ethanol yields of co-fermentation of corn mash and PCS filtrate. Yield in g-ethanol/g-DS is only based on corn mash DS, not including DS from PCS filtrate.

FIG. 2 shows the ethanol concentrations determined by HPLC after 68 hours co-fermentation of PCS filtrate and corn mash, x-PCS indicates volume of PCS filtrate introduced to 5 g-CM.

### DETAILED DESCRIPTION OF THE INVENTION

Methods and processes of the invention may advantageously be applied in situation where lignocellulose based (i.e., biomass) ethanol plants are co-located with, e.g., existing starch based ethanol plants.

### Methods of Producing a Fermentation Product

According to the invention fermentation products are produced by co-fermenting sugars derived from two separate streams, i.e., one stream containing sugars derived from lignocellulose-containing material and another stream containing starch-containing material. In a preferred embodiment the lignocellulose derived sugars are added to the saccharification step and/or fermentation step or simultaneous saccharification and fermentation step in a process of converting starch into a desired fermentation product. The lignocellulose derived sugars may be in the form of liquor (e.g. filtrate) from pre-treated and/or hydrolyzed lignocellulose-containing material.

According to the invention fermentation of starch derived and lignocellulose derived fermentable sugars, such as glucose, is integrated. More specifically mash derived from starch-containing material and pre-treated and/or hydrolyzed lignocellulose-containing material is co-fermentation, preferably in a SSF process. The pre-treated lignocellulose-containing material may be filtrate and may even be unwashed filtrate. For instance, the filtrate may be the liquid phase after a solid-liquid separation of pre-treated and/or hydrolyzed lignocellulose-containing material; e.g., pre-treated and/or hydrolyzed corn stover.

The inventor found that integrating fermentation of especially C6 sugars from pretreated corn stover (PCS liquor/filtrate) with fermentation of corn mash showed no negative impact on the ethanol yield at the end of fermentation. Example 1 illustrates this finding.

Consequently, in the first aspect the invention relates to methods for producing a fermentation product from lignocellulose-containing material, wherein the methods comprise

i) pre-treating lignocellulose-containing material;

ii) introducing pre-treated lignocellulose-containing material into medium comprising fermentable sugars derived from starch-containing material,

iii) fermenting using a fermenting organism.

The medium may be a starch saccharification medium and/or starch fermentation medium. In a preferred embodiment the method comprises introducing pre-treated lignocellulose-containing material into a simultaneous saccharification and fermentation medium containing one or more starch-degrading enzymes and optionally a fermenting organism.

The actual fermentation may be initiated before or after the pre-treated lignocellulose material is introduced into the fermentation medium. The pre-treated lignocellulose-containing material is preferably hydrolyzed before it is added to the co-fermentation step, i.e., saccharification step, fermentation step or simultaneous saccharification and fermentation step.

In a preferred embodiment solids (comprising mainly lignin and unconverted polysaccharides) are removed from the pre-treated and/or hydrolyzed lignocellulose-containing material stream before and/or during saccharification, fermentation or simultaneous saccharification and fermentation. In other words, the lignocellulose-containing material stream having solids removed is added during starch saccharification, fermentation and/or simultaneous saccharification and fermentation. The solids may be removed in any suitable way know in the art. In suitable embodiments the solids can be removed by filtration, or by using a filter press and/or centrifuge, or the like. As mentioned above one of the advantages of the invention is that the pre-treated lignocellulose-containing material may be un-detoxified, i.e., the pre-treated lignocellulose-containing material may contain compounds that could be toxic to the fermenting organism. Further, the pre-treated lignocellulose-containing material may also contain compounds that could inactivate or at least significantly inhibit the performance of enzymes, e.g., cellulases, hemicellulases and/or other enzymes involved in hydrolyzing the pre-treated material into sugars, and/or starch-degrading enzymes used for converting starch into sugars.

Which toxic and/or inhibitory compounds are present in the lignocellulose derived stream depends to a large extent on the pre-treatment method used and the actual lignocellulose-containing material. Examples of toxic and/or inhibitory compounds, i.e., pre-treated lignocellulose degradation products, include 4-OH benzyl alcohol, 4-OH benzaldehyde, 4-OH benzoic acid, trimethyl benzaldehyde, 2-furoic acid, coumaric acid, ferulic acid, phenol, guaiacol, veratrole, pyrogallollol, pyrogallol mono methyl ether, vanillyl alcohol, isovanillin, vanillic acid, isovanillic acid, homovanillic acid, veratryl alcohol, veratraldehyde, veratric acid, 2-O-methyl gallic acid, syringyl alcohol, syringaldehyde, syringic acid, trimethyl gallic acid, homocatechol, ethyl vanillin, creosol, p-methyl anisol, anisaldehyde, anisic acid, furfural, hydroxymethylfurfural, 5-hydroxymethylfurfural, formic acid, acetic acid, levulinic acid, cinnamic acid, coniferyl aldehyde, isoeugenol, hydroquinone, and eugenol.

According to the invention starch-containing material and lignocellulose-containing material are treated in two separate streams before combining the streams during starch saccharification, fermentation or simultaneous saccharification and fermentation. When the streams are combined the lignocellulose derived material may constitute from 0.1 to 90 wt. %, preferably 1 to 80 wt. %, such as 10 to 70 wt. %, especially 20 to 60 wt. %, such as around 50 wt. % of the total weight of the combined fermentation medium.

The pre-treated lignocellulose derived material introduced into the saccharification, fermentation, or simultaneous saccharification and fermentation medium may be un-washed. Pre-treated lignocellulose-containing material is normally washed, or detoxified in another way, in order to removed unwanted toxic compounds, but according to the invention this is not required. In a specific embodiment the material is un-washed pre-treated and/or hydrolyzed corn stover.

### Lignocellulose-Containing Materials

The term lignocellulose-containing material' means material primarily consisting of cellulose, hemicellulose, and lignin and is often referred to as “biomass”.

The structure of lignocellulose is not directly accessible to enzymatic hydrolysis. Therefore, the lignocellulose has to be pre-treated, e.g., by acid hydrolysis under adequate conditions of pressure and temperature, in order to break the lignin seal and disrupt the crystalline structure of cellulose. This causes solubilization and saccharification of the hemicellulose fraction. The cellulose fraction can then be hydrolyzed enzymatically, e.g., by cellulase enzymes, to convert the carbohydrate polymers into fermentable sugars which may be fermented into a desired fermentation product, such as ethanol, which may optionally be recovered, e.g., by distillation.

The lignocellulose-containing material may be any material containing lignocellulose. In a preferred embodiment the lignocellulose-containing material contains at least 30 wt. 90 preferably at least 50 wt. %, more preferably at least 70 wt. %, even more preferably at least 90 wt. %, lignocellulose. It is to be understood that the lignocellulose-containing material may also comprise other constituents such as cellulosic material, including cellulose and hemicellulose, and may also comprise constituents such as proteinaceous material, starch, and sugars such as fermentable sugars and/or un-fermentable sugars.

Lignocellulose-containing material is generally found, for example, in the stems, leaves, hulls, husks, and cobs of plants or leaves, branches, and wood of trees. Lignocellulose-containing material can also be, but is not limited to, herbaceous material, agricultural residues, forestry residues, municipal solid wastes, waste paper, and pulp and paper mill residues. It is to be understood that lignocellulose-containing material may be in the form of plant cell wall material containing lignin, cellulose, and hemi-cellulose in a mixed matrix.

In a preferred embodiment the lignocellulose-containing material is selected from one or more of corn fiber, rice straw, pine wood, wood chips, poplar, bagasse, and paper and pulp processing waste.

Other examples of suitable lignocellulose-containing material include corn stover, corn cobs, hard wood such as poplar and birch, soft wood, cereal straw such as wheat straw, switch grass, Miscanthus, rice hulls, municipal solid waste (MSW), industrial organic waste, office paper, or mixtures thereof.

In a preferred embodiment the lignocellulose-containing material is corn stover or corn cobs. In another preferred embodiment, the lignocellulose-containing material is corn fiber.

### Pre-Treatment

The lignocellulose-containing material may be pre-treated in any suitable way.

Pre-treatment is carried out before hydrolysis or (co-)fermentation. In a preferred embodiment the pre-treated material is hydrolyzed, preferably enzymatically, before fermentation. The goal of pre-treatment is to separate and/or release cellulose, hemicellulose, and/or lignin to improve the rate of hydrolysis. Pre-treatment methods such as wet-oxidation and alkaline pre-treatment target lignin release, while dilute acid and auto-hydrolysis target hemicellulose release. Steam explosion is an example of a pre-treatment that targets cellulose release.

According to the invention the pre-treatment step may be a conventional pre-treatment step using techniques well known in the art. In a preferred embodiment pre-treatment takes place in aqueous slurry. The lignocellulose-containing material may during pre-treatment be present in an amount between 10-80 wt. %, preferably between 20-70 wt. %, especially between 30-60 wt. %, such as around 50 wt. %.

Chemical Mechanical and/or Biological Pre-Treatment

According to the invention, the lignocellulose-containing material may be pre-treated chemically, mechanically, biologically, or any combination thereof, before hydrolysis. Mechanical pre-treatment (often referred to as “physical” pre-treatment) may be used alone or in combination with subsequent or simultaneous hydrolysis, especially enzymatic hydrolysis.

Preferably, the chemical, mechanical, or biological pre-treatment is carried out prior to the hydrolysis. Alternatively, the chemical, mechanical, or biological pre-treatment may be carried out simultaneously with hydrolysis, such as simultaneously with addition of one or more cellulase enzymes, or other enzyme activities, to release, e.g., fermentable sugars, such as glucose and/or maltose.

In an embodiment of the invention the pre-treated lignocellulose-containing material may be washed or detoxified. However, washing and detoxification is not mandatory and is in a preferred embodiment eliminated. In a preferred embodiment the pre-treated lignocellulose-containing material is unwashed or un-detoxified.

### Chemical Pre-Treatment

The term “chemical treatment” refers to any chemical pre-treatment which promotes the separation or release of cellulose, hemicellulose, or lignin. Examples of suitable chemical pre-treatments include treatment with one or more of, for example, dilute acid, lime, alkaline, organic solvent, ammonia, sulfur dioxide, and carbon dioxide. Further, wet oxidation and pH-controlled hydrothermolysis are also considered chemical pre-treatment.

In a preferred embodiment the chemical pre-treatment is acid treatment. More preferably, the chemical pre-treatment is a continuous dilute and/or mild add treatment such as treatment with sulfuric acid, or another organic acid such as acetic acid, citric add, tartaric acid, succinic acid, hydrogen chloride or mixtures thereof. Other acids may also be used. Mild acid treatment means that the treatment pH lies in the range from pH 1-5, and preferably pH 1-3. In a specific embodiment the acid concentration is in the range from 0.1 to 2.0 wt, % add, and is preferably sulphuric acid. The acid may be contacted with the lignocellulose-containing material and the mixture may be held at a temperature in the range of 160-220° C., such as 165-195° C., for periods ranging from minutes to seconds, e.g., 1-60 minutes, such as 2-30 minutes or 3-12 minutes. Addition of strong acids, such as sulphuric acid, may be applied to remove hemicellulose. Such strong acids enhance the digestibility of cellulose.

Other chemical pre-treatment techniques are also contemplated. Cellulose solvent treatment has been shown to convert about 90% of cellulose to glucose. It has also been shown that enzymatic hydrolysis could be greatly enhanced when the lignocellulose structure is disrupted. Alkaline H₂O₂, ozone, organosolv (using Lewis acids, FeCl₃, (Al)₂SO₄ in aqueous alcohols), glycerol, dioxane, phenol, or ethylene glycol are among solvents known to disrupt cellulose structure and promote hydrolysis (Mosier et al., 2005, Bioresource Technology 96: 673-686).

Alkaline chemical pre-treatment with base, e.g., NaOH, Na₂CO₃ or ammonia or the like, is also contemplated according to the invention. Pre-treatment methods using ammonia are described in, e.g. WO 2006/110891, WO 2006/110899, WO 2006/110900, WO 2006/110901, which are hereby incorporated by reference.

Wet oxidation techniques involve use of oxidizing agents such as sulphite based oxidizing agents or the like. Examples of solvent pre-treatments include treatment with DMSO (dimethyl sulfoxide) or the like. Chemical pre-treatment is generally carried out for 1 to 60 minutes, such as from 5 to 30 minutes, but may be carried out for shorter or longer periods of time dependent on the material to be pre-treated.

Other examples of suitable pre-treatment methods are described by Schell et al., 2003, Appl. Biochem and Biotechn. 105-108: 69-85, and Mosier et al., 2005, Bioresource Technology 96: 673-686, and U.S. Application Publication No. 2002/0164730, which references are hereby incorporated by reference.

### Mechanical Pre-Treatment

The term “mechanical pre-treatment” refers to any mechanical for physical) pre-treatment which promotes the separation or release of cellulose, hemicellulose, or lignin from lignocellulose-containing material. For example, mechanical pre-treatment includes various types of milling, irradiation, steaming/steam explosion, and hydrothermolysis.

Mechanical pre-treatment includes comminution (i.e., mechanical reduction of the size). Comminution includes dry milling, wet milling and vibratory ball milling. Mechanical pre-treatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is a batch-process steam gun hydrolyzer system which uses high pressure and high temperature as defined above. A Sunds Hydrolyzer (available from Sunds Defibrator AB (Sweden)) may be used for this.

### Combined Chemical and Mechanical Pre-Treatment

In a preferred embodiment both chemical and mechanical pre-treatments are carried out. For instance, the pre-treatment step may involve dilute or mild acid treatment and high temperature and/or pressure treatment. The chemical and mechanical pre-treatments may be carried out sequentially or simultaneously, as desired.

Accordingly, in a preferred embodiment, the lignocellulose-containing material is subjected to both chemical and mechanical pre-treatment to promote the separation or release of cellulose, hemicellulose, or lignin.

In a preferred embodiment the pre-treatment is carried out as a dilute and/or mild acid steam explosion step. In another preferred embodiment pre-treatment is carried out as an ammonia fiber explosion step (or AFEX pre-treatment step).

### Biological Pre-Treatment

The term “biological pre-treatment” refers to any biological pre-treatment which promotes the separation or release of cellulose, hemicellulose, or lignin from the lignocellulose-containing material. Biological pre-treatment techniques can involve applying lignin-solubilizing microorganisms (see, for example, Hsu, 1996, Pretreatment of biomass, in Handbook on Bioethanol: Production and Utilization, Wyman, C. E., ed., Taylor & Francis, Washington, D.C., 179-212; Ghosh and Singh, 1993, Physicochemical and biological treatments for enzymatic/microbial conversion of lignocellulosic biomass, Adv. Appl. Microbiol. 39: 295-333; McMillan, 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech, 18: 312-331; and Vallander and Eriksson, 1990. Production of ethanol from lignocellulosic materials: State of the art, Adv. Biochem. Eng./Biotechnol. 42: 63-95),

### Hydrolysis

Before the pre-treated lignocellulose-containing material is added/introduced/combined into starch the saccharification, fermentation or simultaneous saccharification and fermentation step, it may be hydrolyzed to break down cellulose and hemicellulose.

The dry solids content during hydrolysis may be in the range from 5-50 wt. %, preferably 10-40 wt, %, preferably 20-30 wt. %. Hydrolysis may in a preferred embodiment be carried out as a fed batch process where the pre-treated lignocellulose-containing material (substrate) is fed gradually to, e.g., an enzyme containing hydrolysis solution.

In a preferred embodiment hydrolysis is carried out enzymatically. According to the invention the pre-treated lignocellulose-containing material may be hydrolyzed by one or more hydrolases (class EC 3 according to Enzyme Nomenclature), preferably one or more carbohydrases selected from the group consisting of cellulase, hemicellulase, amylase such as alpha-amylase, and carbohydrate-generating enzyme such as glucoamylase, proteases, and esterase such as lipase. For instance, alpha-amylase, glucoamylase and/or the like may be present during hydrolysis and/or fermentation as the lignocellulose-containing material may include some starch.

The enzyme(s) used for hydrolysis is(are) capable of directly or indirectly converting carbohydrate polymers into fermentable sugars which can be fermented into a desired fermentation product such as ethanol.

In a preferred embodiment the carbohydrase has cellulase activity. Suitable carbohydrases are described in the “Enzymes” section below.

Hemicellulose polymers can be broken down by hemicellulases and/or acid hydrolysis to release its five and six carbon sugar components. The six carbon sugars (hexoses), such as glucose, galactose, arabinose, and mannose, can readily be fermented to, e.g., ethanol, acetone, butanol, glycerol, citric acid, fumaric acid etc. by suitable fermenting organisms including yeast. Preferred for ethanol fermentation is yeast, such as of the genus Saccharomyces, especially of the species Saccharomyces cerevisiae, preferably strains which are resistant towards high levels of ethanol, i.e., up to, e.g., above 10, 12 or 15 vol. % ethanol or more, such as above 20 vol. % ethanol.

In a preferred embodiment the pre-treated lignocellulose-containing material is hydrolyzed using a hemicellulase, preferably a xylanase, esterase, cellobiase, or combination thereof.

Hydrolysis may also be carried out in the presence of a combination of hemicellulase(s) and/or cellulase(s), and optionally one or more of the other enzymes mentioned in the “Enzyme” section below.

In an embodiment xylose isomerase may be used for hydrolyzing pre-treated lignocellulose-containing material. Xylose isomerase can convert xylose to xylulose that can be fermented by fermenting organisms like Saccharomyces to a desired fermentation product. Consequently, in an embodiment a xylose isomerase is added during hydrolysis.

Enzymatic treatment may be carried out in a suitable aqueous environment under conditions which can readily be determined by one skilled in the art. In a preferred embodiment hydrolysis is carried out at suitable, preferably optimal, conditions for the enzyme(s) in question.

Suitable process time, temperature and pH conditions can readily be determined by one skilled in the art. Preferably, hydrolysis is carried out at a temperature between 25 and 70° C., preferably between 40 and 60° C., especially around 50° C. The process is preferably carried out at a pH in the range from pH 3-8, preferably pH 4-6, especially around pH 5. Hydrolysis is typically carded out for between 12 and 96 hours, preferable 16 to 72 hours, more preferably between 24 and 48 hours.

### Fermentation

According to the invention sugars from pre-treated and/or hydrolyzed lignocellulose-containing material are co-fermented together with sugars obtained from starch-containing material using at least one fermenting organism capable of fermenting fermentable sugars, such as glucose, xylose, mannose, and galactose directly or indirectly into a desired fermentation product. The fermentation conditions depend on the desired fermentation product and can easily be determined by one of ordinary skill in the art.

In the case of ethanol fermentation with yeast the fermentation is preferably ongoing for between 1-120 hours, preferably 12-96 hours. In an embodiment the fermentation is carried out at a temperature between 20 to 40″C, preferably 26 to 34° C. in particular around 32° C. In an embodiment the pH is from pH 3-7, preferably pH 4-6.

In a preferred embodiment (co-)fermentation is carried out as a starch simultaneous saccharification and fermentation (SSF) process, and the lignocellulose derived (sugar) stream, preferably unwashed filtrate, is added/introduced/combined into the starch SSF process. In other words, in a preferred embodiment there is no separate saccharification step, meaning that the fermenting organism(s) and starch-degrading enzyme(s), such as carbohydrate-source generating enzyme(s), are added together,

### Recovery

Subsequent to fermentation the fermentation product may optionally be separated from the fermentation medium in any suitable way. For instance, the medium may be distilled to extract the fermentation product or the fermentation product may be extracted from the fermentation medium by micro or membrane filtration techniques. Alternatively the fermentation product may be recovered by stripping. Recovery methods are well known in the art.

### Fermentation Products

The present invention may be used for producing any fermentation product. Preferred fermentation products include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, 812, beta-carotene); and hormones.

Other products include consumable alcohol industry products, e.g., beer and wine; dairy industry products, e.g., fermented dairy products; leather industry products and tobacco industry products. In a preferred embodiment the fermentation product is an alcohol, especially ethanol. The fermentation product, such as ethanol, obtained according to the invention, may preferably be used as fuel alcohol/ethanol. However, in the case of ethanol it may also be used as potable ethanol.

### Fermenting Organisms

The term “fermenting organism” refers to any organism, including bacterial and fungal organisms such as yeast and filamentous fungi, suitable for producing a desired fermentation product. Especially suitable fermenting organisms are able to ferment, i.e. convert, sugars, such as glucose, fructose, maltose, xylose, mannose and or arabinose, directly or indirectly into the desired fermentation product. Examples of fermenting organisms include fungal organisms such as yeast. Preferred yeast includes strains of the genus Saccharomyces, in particular strains of Saccharomyces cerevisiae or Saccharomyces uvarum: a strain of Pichia, preferably Pichia stipitis such as Pichia stipitis CBS 5773 or Pichia pastoris; a strain of the genus Candida, in particular a strain of Candida utilis, Candida arabinofermentans, Candida diddensii, Candida sonorensiS, Candida shehatae, Candida tropicalis, or Candida boidinii. Other fermenting organisms include strains of Zymomonas; Hansenula, in particular Hansenula polymorpha or Hansenula anomala: Kluyveromyces, in particular Kluyveromyces fragilis or Kluyveromyces; and Schizosaccharomyces, in particular Schizosaccharomyces pombe.

Preferred bacterial fermenting organisms include strains of Escherichia, in particular Escherichia coli, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes and strains of Thermoanaerobacter, in particular Thermoanaerobacter BG1L1 (Appl. Microbiol, Biotech. 77: 61-86) and Thermoanarobacter ethanolicus, Thermoanaerobacter thermosaccharolyticum, or Thermanaerobacter mathranii, Strains of Lactobacillus are also envisioned as are strains of Corynebacterium glutamicum R, Bacillus thermoglucosidaisus, and Geobacillus thermoglucosidasius.

In an embodiment the fermenting organism is a C6 sugar fermenting organism, such as a strain of, e.g., Saccharomyces cerevisiae.

In connection with fermentation of lignocellulose derived materials, C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp. that are capable of fermenting C5 sugars include the ones concerned in, e.g., Ho et al., 1998, Applied and Environmental Microbiology, p. 1852-1859 and Karhumaa et al., 2006, Microbial Cell Factories 518.

In one embodiment the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

Commercially available yeast includes, e.g.; RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA); GERT STRAND (available from Gert Strand AB, Sweden), and FERMIOL (available from DSM Specialties).

### Starch-Containing Materials

The starch-containing material may be any suitable starch-containing material.

As indicated below, the starch may be either liquefied gelatinized starch or un-gelatinized starch (e.g. uncooked granular starch).

The actual starting material is generally selected based on the desired fermentation product. Examples of starch-containing materials suitable for use in a method or process of present invention include tubers, roots, stems, whole grains, corns; cobs, wheat; barley, rye, milo, sago, cassaya, tapioca: sorghum, rice peas, beans, or sweet potatoes, or mixtures thereof, or cereals, sugar-containing raw materials, such as molasses, fruit materials, sugar cane or sugar beet, potatoes or mixtures thereof. Contemplated are both waxy and non-waxy types of corn and barley.

The term “granular starch” means raw uncooked starch. i.e., starch in its natural form found in, e.g. cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may be a highly refined starch quality, preferably at least 90%, at least 95%, at least 97% or at least 99.5% pure or it may be a more crude starch containing material comprising milled whole grain including non-starch fractions such as germ residues and fibers.

### Fractionation of Starch-Containing Material

In an embodiment the starch-containing material is fractionated into one or more components, including fiber, germ, and a mixture of starch and protein (endosperm). Fractionation may according to the invention be done using any suitable technology or apparatus. For instance, Satake Corporation (Japan) has manufactured a system suitable for fractionation of plant material such as corn.

The germ and fiber components may be fractionated from the remaining potion of the endosperm. In an embodiment of the invention the starch-containing material is plant endosperm, preferably corn endosperm. Further, the endosperm may be reduced in particle size and combined with the larger pieces of the fractionated germ and fiber components for fermentation.

Fractionation can be accomplished by using an apparatus such as, e.g., the one disclosed in U.S. Application Publication No, 2004/0043117, which is hereby incorporated by reference to the extent it teaches an apparatus and its use for fractionation. Suitable methods and apparatus for fractionation include a sieve, sieving and elutriation. Suitable apparatus also include friction mills, such as rice or grain polishing mills (e.g., those manufactured by Satake Corporation (Japan), Kett, or Rapsco, Tex., USA).

### Reducing the Particle Size of Starch-Containing Material

The starch-containing material may preferably be reduced in particle size in order to open up the structure and expose more surface area. This may be done by milling. Two milling processes are preferred according to the invention: wet and dry milling. In dry milling whole kernels are milled and used. Wet milling gives a good separation of germ and meal (i.e., starch granules and protein). Both dry and wet milling is well known in the art of starch processing and is equally contemplated according to the invention. Examples of other contemplated technologies for reducing the particle size of the starch-containing material include emulsifying technology and rotary pulsation.

The starch-containing material may in one embodiment be reduced in particle size to between 0.05 to 3.0 mm, or so that at least 30%, preferably at least 50%, more preferably at least 70%, even more preferably at least 90% of the starch-containing material fit through a sieve with a 0.05 to 3.0 mm screen, preferably 0.1 to 0.5 mm screen.

Processes for Producing a Fermentation Product by Adding Pre-Treated Lignocellulose-Containing Material into a Conventional Starch Based Process

In this aspect the invention relates to a process for producing a fermentation product from a combination of starch-containing material and lignocellulose-containing material comprising the steps of:

a) liquefying starch-containing material;

b) saccharifying; and

c) fermenting using a fermenting organism;

wherein the pre-treated lignocellulose-containing material is added before and/or during fermentation.

It is to be understood that the starch-containing material and lignocellulose-containing material are treated in two separate streams before being combined. In a preferred embodiment the lignocellulose derived material is introduced into fermentation so that it constitutes from 0.1 to 90 wt. %, preferably 1 to 80 wt. %, such as 10 to 70 wt. % especially 20 to 60 wt. %, such as around 50 wt. % of the total weight of the combined fermentation medium.

In a preferred embodiment step a) is carried out in the presence of one or more alpha-amylases. The alpha-amylase(s) may preferably be of bacterial or fungal origin. Examples of alpha-amylases are described in the “Alpha-Amylases” section below.

Further, saccharification step b) or simultaneous steps b) and c) (i.e., SSF), are preferably carried out in the presence of one or more carbohydrate-source generating enzyme such as especially a glucoamylase. Fermentation step c) or simultaneous steps b) and c) are preferably carried out in the presence of yeast, preferably a strain of Saccharomyces, such as a strain of Saccharomyces cerevisae. Suitable fermenting organisms are listed in the “Fermenting Organisms” section above.

The desired fermentation product is ethanol. The fermentation product, especially ethanol, may optionally be recovered after fermentation, e.g., by distillation.

Suitable starch-containing starting materials are listed in the section ‘Starch-Containing Materials’ section above. Contemplated enzymes are listed in the “Enzymes” section below.

In a particular embodiment the process further comprises, prior to the step a), the steps of:

1) reducing the particle size of the starch-containing material, preferably by milling; and

2) forming a slurry comprising the starch-containing material and water.

The aqueous slurry may contain from 10 to 55 wt, %, preferably 25 to 40 wt, %, more preferably 30 to 35 wt. % starch-containing material. In this aspect of the invention the slurry is heated to above the gelatinization temperature. Optionally alpha-amylase may be added at this point in time to initiate liquefaction (thinning). The slurry may then be jet-cooked to further gelatinize the slurry before being subjected to alpha-amylase in step a).

More specifically liquefaction may be carried out as a three-step hot slurry process. The slurry is heated to between 60-105° C., preferably 80-95° C., and alpha-amylase may be added to initiate liquefaction (thinning). In one embodiment the slurry is then jet-cooked at a temperature between 95-140° C., preferably 105-125° C., for 1-15 minutes, preferably for 3-10 minutes, especially around 5 minutes. The slurry is cooled to 60-105° C. and (more) alpha-amylase is added to finalize hydrolysis (secondary liquefaction), The liquefaction step may be carried out at a pH from 3-7, in particular at a pH between 4-6, especially at a pH between 4-5.

Saccharification in step b) may be carried out using conditions well known in the art. For instance, a full, separate saccharification step may last up to from about 24 to about 72 hours. In one embodiment a pre-saccharification of about 40-90 minutes at a temperature between 30-65° C., typically about 60° C., is carried out, followed by complete saccharification during fermentation in a simultaneous saccharification and fermentation step (i.e., SSF). Saccharification is typically carried out at temperatures from 30-65° C., typically around 60° C. and at a pH between 4 and 5, normally at about pH 4.5.

The most widely used step in fermentation product, especially ethanol, production is a simultaneous saccharification and fermentation (SSF) step, in which there is no holding stage for the saccharification, meaning that the fermenting organism, such as yeast, and enzyme(s) may be added together. SSF may typically be carried out at a temperature between 25° C. and 40° C., such as between 29° C. and 35° C., such as between 30° C. and 34° C., such as around 32° C. In other words, saccharification in step b) and fermentation in step c) may be carried out either sequentially or simultaneously, preferably simultaneously.

In a preferred embodiment the pre-treated lignocellulose-containing material is hydrolyzed before it is added to the starch saccharification, fermentation or simultaneous saccharification and fermentation step. Suitable pre-treatment methods are described above in the section “Pre-treatment” above.

The pre-treated lignocellulose material may further be hydrolyzed by treatment with one or more hydrolases (class EC 3 according to Enzyme Nomenclature), preferably one or more carbohydrases, such as cellulase or hemicellulase, or a combination thereof, before fermentation. Examples of suitable hydrolases can be found below.

Solids from the pre-treated and/or hydrolyzed lignocellulose-containing material are preferably removed before fermentation. Therefore, the pre-treated and/or hydrolyzed lignocellulose-containing material having solids removed are added to the saccharification step b), fermentation step c), or simultaneous saccharification and fermentation step. The solids from the pre-treated lignocellulose-containing material may be removed in any suitable way known in the art. For instance, solids may be removed by filtration, use of a filter press and/or centrifuge, or the like. As also mentioned above the pre-treated lignocellulose-containing material may be un-detoxified, such as un-washed.

One or more carbohydrate-generating enzymes may be used during saccharification, fermentation or simultaneous saccharification and fermentation. Examples of such enzymes are disclosed in the “Carbohydrate-Source Generating Enzymes” section below. The preferred carbohydrate-source generating enzyme is a glucoamylase.

### Processes for Producing a Fermentation Product by Adding Pre-Treated Lignocelluiose-Containing Material to a Process of Fermenting Un-Cooked Starch Based Material

In this aspect the invention relates to processes for producing a fermentation product from a combination of starch-containing material and pre-treated lignocellulose-containing material. The process is carried out without cooking of the starch-containing material (Le., no gelatinization occurs). In other words, according to this aspect of the invention the desired fermentation product is produced without liquefying the slurry containing the starch-containing material. In one embodiment a process of the invention includes saccharifying (e.g., milling) starch-containing material, preferably granular starch, below the initial gelatinization temperature in the presence of either an alpha-amylase as exemplified in the “Alpha-Amylase” section below and/or a carbohydrate-source generating enzyme, preferably a glucoamylase, exemplified in the “Carbohydrate-Source Generating Enzymes” section below, to produce sugars that can be fermented and converted into a desired fermentation product by one or more suitable fermenting organisms.

Consequently, in this aspect the invention relates to processes for producing a fermentation product from a combination of starch-containing material and lignocellulose-containing material comprising the steps of:

i) saccharifying starch-containing material at a temperature below the initial gelatinization temperature:

ii) fermenting using a fermenting organism;

wherein the pre-treated lignocellulose containing material is added before and/or during fermentation.

It is to be understood that the starch-containing material and lignocellulose-containing material are treated in two separate streams before being combined. In a preferred embodiment the lignocellulose derived material is introduced into fermentation so that it constitutes from 0.1 to 90 wt. %, preferably 1 to 80 wt. %, such as 10 to 70 wt. %, especially 20 to 60 wt. %, such as around 50 wt, % of the total weight of the (combined) fermentation medium.

Optionally the fermentation product is recovered after fermentation. The saccharification in step i) and fermentation in step ii) may be carried out either sequentially or simultaneously, preferably simultaneously. In a preferred embodiment the pre-treated lignocellulose-containing material is hydrolyzed before being added to the saccharification step, fermentation step or simultaneous saccharification and fermentation step. The pre-treated material has in a preferred embodiment been hydrolyzed by treatment with one or more hydrolases (class EC 3 according to Enzyme Nomenclature), preferably one or more carbohydrases, preferably cellulase(s) or hemicellulase(s), or a combination thereof, before fermentation. Examples of other hydrolyses can be found below.

Solids from the pre-treated and/or hydrolyzed lignocellulose-containing material are preferably removed before added to saccharification, fermentation or simultaneous saccharification and fermentation. Therefore, the pre-treated and/or hydrolyzed lignocellulose-containing material having solids removed may according to the invention be added to the saccharification step i), fermentation step ii), or simultaneous saccharification and fermentation step. The solids from the pre-treated lignocellulose-containing material may be removed in any suitable way. For instance, solids may be removed by filtration, use of a filter press and/or by centrifugation, or the like. As also mentioned above the pre-treated lignocellulose-containing material may be un-detoxified, such as un-washed. One or more carbohydrate-generating enzymes may be used during saccharification, fermentation or simultaneous saccharification and fermentation. The lignocellulose-containing material may be pre-treated in any suitable way before being added to the saccharification step, fermentation step or simultaneous saccharification and fermentation step. Examples of chemical, mechanical and/or biological pre-treated methods are disclosed above in the “Pre-treatment” section.

The desired fermentation product is preferably ethanol. Examples of other desired fermentation products can be found in the “Fermentation Products” section above.

For ethanol production the preferred fermenting organism is yeast, preferably a strain of the genus Saccharomyces. Examples of other fermenting organisms can be found in the “Fermentation Organisms” section above. Suitable process conditions are well known in the art. In a preferred embodiment the fermentation or simultaneous saccharification and fermentation is carried out at a temperature between 25° C. and 40° C., such as between 29° C. and 35° C., such as between 3CPC and 34′.C, such as around 32° C. The pH during fermentation may suitably be between 3 and 7, preferably between 4 and 6. Fermentation may be carded out for 1-120 hours, preferably 12-96 hours. Examples of suitable lignocellulose-containing materials and starch-containing materials can be found above.

The phrase “below the initial gelatinization temperature.” means below the lowest temperature at which gelatinization of the starch in question commences. Starch heated in water begins to gelatinize between about 50° C. and 75° C. The exact temperature of gelatinization depends on the specific starch and can readily be determined by the skilled artisan. Thus, the initial gelatinization temperature may vary according to the plant species, to the particular variety of the plant species as well as with the growth conditions. In the context of this invention the initial gelatinization temperature of a given starch-containing material can be defined as the temperature at which birefringence is lost in 5% of the starch granules using the method described by Gorinstein and Lii, 1992, Starch/Starke, 44 (12): 461-466.

Before step i) a slurry of starch-containing material, such as granular starch, having between 10 to 55 wt, % dry solids (DS), preferably between 25 to 40 wt. % dry solids, more preferably 30 to 35 wt. % dry solids of starch-containing material, may be prepared. The pre-treated and/or hydrolyzed lignocellulose-containing material may be added at this point in time. The slurry may also include water and/or process water, such as thin stillage (backset), scrubber water, evaporator condensate or distillate, side stripper water from distillation, or other fermentation product plant process water. The composition of the slurry can easily be adjusted by one skilled in the art.

The starch-containing material may be prepared by reducing the particle size, preferably by dry or wet milling, to between 0.05 to 3.0 mm, preferably between 0.1 to 0.5 mm. After being subjected to a process of the invention at least 60%, at least 70%, at least 80%, at least 90%, at least 90%, at least 91%, at least 92%, at least 93%, at least 94%, at least 95%, at least 96%, at least 97%, at least 98%, or preferably at least 99% of the dry solids of the starch-containing material is converted into a soluble starch hydrolysate.

According to this aspect of the invention the process is conducted at a temperature below the initial gelatinization temperature. In the case where saccharification in step i) and fermentation is step ii) is carried out sequentially the temperature may typically be between 30-75° C., preferably between 45-60° C. In a preferred embodiment step i) and step ii) are carried out simultaneously. Suitable conditions are described above.

In a preferred embodiment the sugar level, such as glucose level, is kept at a low level such as below 6 wt. %, preferably below about 3 wt. %, preferably below about 2 wt. %, more preferred below about 1 wt. %, even more preferred below about 0.5 wt, %, or even more preferred 0.25% wt. %, such as below about 0.1 wt. %. Such low levels of sugar can be accomplished by simply employing adjusted quantities of enzyme and fermenting organism. A skilled person in the art can easily determine which quantities of enzyme and fermenting organism to use, The employed quantities of enzyme and fermenting organism may also be selected to maintain low concentrations of maltose in the fermentation medium. For instance, the maltose level may be kept below about 0.5 wt. % or below about 0.2 wt. %.

### Enzymes

Even if not specifically mentioned in context of a method or process of the invention, it is to be understood that the enzyme(s) as well as other compounds are used in an effective amount.

### Cellulases

The term “cellulases” as used herein are understood as comprising the cellobiohydrolases (EC 3.2.1.91), e.g., cellobiohydrolase I and cellobiohydrolase II, as well as the endo-glucanases (EC 3.2.1.4), The phrase “cellulolytic enzymes” as used herein is understood as including cellobiohydrolases (EC 3.2.1.91), e.g. cellobiohydrolase I and cellobiohydrolase II, as well as endo-glucanases (EC 3.2.1.4) and beta-glucosidases (EC 3.2.1.21).

In order to be efficient, the digestion of cellulose and hemicellulose requires several types of enzymes acting cooperatively. At least three categories of enzymes are necessary to convert cellulose into fermentable sugars: endo-glucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose, The term “cellobiohydrolase I” is defined herein as a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

Examples of cellobiohydroloses are mentioned above including CBH I and CBH II from Trichoderma reseei: Humicola insolens and CBH II from Thielavia terrestris cellobiohydrolase (CELL6A)

Cellobiohydrolase activity may be determined according to the procedures described by Lever et al., 1972, Anal. Biochem, 47: 273-279 and by van Tilbeurgh at, 1982, FEBS Letters 149: 152-156, van Tilbeurgh and Claeyssens, 1985, FEBS Letters 187: 283-288, The Lever et al. method is suitable for assessing hydrolysis of cellulose in corn stover and the method of van Tilbeurgh at is suitable for determining the cellobiohydrolase activity on a fluorescent disaccharide derivative.

Endoglucanases (EC No. 3.2.1.4) catalyze endo hydrolysis of 1.4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts. The authorized name is endo-1,4-beta-D-glucan 4-glucano hydrolase, but the abbreviated term endoglucanase is used in the present specification. Endoglucanase activity may be determined using carboxymethyl cellulose (CMC) hydrolysis according to the procedure of Ghose, 1987, Pure and App. Chem. 59: 257-268.

In a preferred embodiment endoglucanases may be derived from a strain of the genus Trichoderma, preferably a strain of Trichoderma reesei, a strain of the genus Humicola, such as a strain of Hum/cola insolens; or a strain of Chrysosporium, preferably a strain of Chrysosporium lucknowense.

The cellulases may comprise a carbohydrate-binding module (CBM) which enhances the binding of the enzyme to a cellulose-containing fiber and increases the efficacy of the catalytic active part of the enzyme. A CBM is defined as contiguous amino acid sequence within a carbohydrate-active enzyme with a discreet fold having carbohydrate-binding activity. For further information of CBMs see the CAZy Internet server (supra) or Tomme et al., 1995, in Enzymatic Degradation of Insoluble Polysaccharides (Saddler, J. N. & Penner, M., eds.), Cellulose-binding domains: classification and properties. pp. 142-163, American Chemical Society, Washington.

In a preferred embodiment the cellulase may be a composition as defined in U.S. Application No. 60/941,251, which is hereby incorporated by reference. Specifically, in one embodiment is the cellulase composition used in Example 1 (Cellulase preparation A). In a preferred embodiment the cellulolytic preparation comprising a polypeptide having cellulolytic enhancing activity (GH61A), is preferably Thermoascus aurantiacus GH61A disclosed in WO 2005/074656 (hereby incorporated by reference). The cellulolytic preparation may further comprise a beta-glucosidase, such as a beta-glucosidase derived from a strain of the genus Trichoderma, Aspergillus or Penicillium, including the Humicola insolens CEL45A endoglucanase core/Aspergillus oryzae beta-glucosidase fusion protein disclosed in U.S. application Ser. No. 11/781,151 or PCT/US2007/074038 (Novozymes). In an embodiment the cellulolytic preparation may also comprises a CBH II, preferably Thielavia terrestris cellobiohydrolase II (CEL6A). In an embodiment the cellulolytic preparation also comprises a cellulase enzymes preparation, preferably the one derived from Trichoderma reesei.

The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi at al., 2002, J. Basic Microbiol. 42: 55-66, except different conditions were employed as described herein. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN® 20.

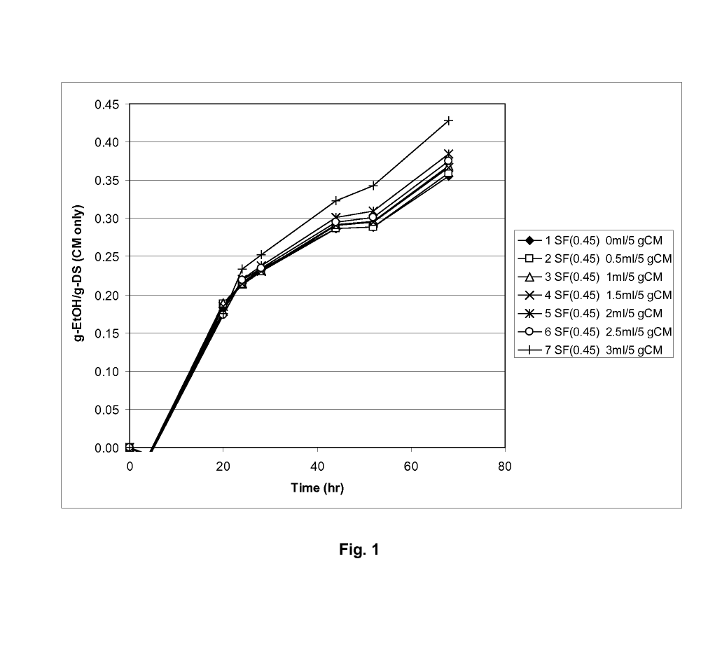

In a preferred embodiment the beta-glucosidase is of fungal origin, such as a strain of the genus Trichoderma, Aspergillus or Penicillium. In a preferred embodiment the beta-glucosidase is a derived from Trichoderma reesei, such as the beta-glucosidase encoded by the bgl1 gene (see FIG. 1 of EP 562003). In another preferred embodiment the beta-glucosidase is derived from Aspergillus oryzae (recombinantly produced in Aspergillus oryzae according to WO 02/095014), Aspergillus fumigatus (recombinantly produced in Aspergillus oryzae according to Example 22 of WO 02/095014) or Aspergillus niger (1981, J. Appl. 3: 157-163).

The cellulolytic activity may, in a preferred embodiment, be derived from a fungal source, such as a strain of the genus Trichoderma, preferably a strain of Trichoderma reesei: or a strain of the genus Humicola, such as a strain of Humicola insolens; or a strain of Chrysosporium, preferably a strain of Chrysosporium lucknowense.

In an embodiment the cellulolytic enzyme preparation comprises a polypeptide having cellulolytic enhancing activity (GH61A) disclosed in WO 2005/074656; a cellobiohydrolase, such as Thielavia terrestris cellobiohydrolase II (CEL6A), a beta-glucosidase (e.g., the fusion protein disclosed in U.S. Application No, 60/832,511) and cellulolytic enzymes, e.g., derived from Trichoderma reesei.

In an embodiment the cellulolytic enzyme preparation comprises a polypeptide having cellulolytic enhancing activity (GH61A) disclosed in WO 2005/074656; a beta-glucosidase (e.g., the fusion protein disclosed in U.S. Application No. 60/832,511) and cellulolytic enzymes, e.g., derived from Trichoderma reesei.

In an embodiment the cellulolytic enzyme is the commercially available product CELLUCLAST® 1.5L or CELLUZYME™ available from Novozymes A/S, Denmark or ACCELERASE™ 1000 (from Genencor Inc., USA).

A cellulolytic enzyme may be added for hydrolyzing the pre-treated lignocellulose-containing material. The cellulolytic enzyme may be dosed in the range from 0.1-100 FPU per gram total solids (TS), preferably 0.5-50 FPU per gram TS, especially 1-20 FPU per gram TS. In another embodiment at least 0.1 mg cellulolytic enzyme per gram total solids (TS), preferably at least 3 mg cellulolytic enzyme per gram TS, such as between 5 and 10 mg cellulolytic enzyme(s) is(are) used for hydrolysis.

### Cellulolytic Enhancing Activity

The phrase “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose-containing material by cellulolytic protein under the following conditions: 1-50 mg of total protein/g of cellulose in PCS (pre-treated corn stover), wherein total protein is comprised of 80-99.5% w/w cellulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide, WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Application Publication No. 2007/0077630 discloses an isolated polypeptide having cellulolytic enhancing activity and a polynucleotide thereof from Trichoderma reesei.

### Hemicellulases

In an embodiment of the invention the pre-treated lignocellulosic material is treated with one or more hemicellulases.

Any hemicellulase suitable for use in hydrolyzing hemicellulose into xylose may be used. Preferred hemicellulases include xylanases, arabinofuranosidases, acetyl xylan esterase, feruloyl esterase, glucuronidases, endo-galactanase, mannases, endo or exo arabinases, exo-galactanses, and mixtures of two or more thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes A/S, Denmark).

In an embodiment the hemicellulase is a xylanase. In an embodiment the xylanase may preferably be of microbial origin, such as of fungal origin (e.g., Trichoderma, Meriplus, Humicola, Aspergillus, Fusarium) or from a bacterium (e.g., Bacillus), In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes NS, Denmark.

The hemicellulase may be added in an amount effective to hydrolyze hemicellulose into xylose, such as, in amounts from about 0001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate,

### Alpha-Amylases

According to the invention any alpha-amylase may be used. Preferred alpha-amylases are of microbial, such as bacterial or fungal origin. Which alpha-amylase is the most suitable depends on the process in question (e.g., gelatinized or un-gelatinized starch) and can easily be determined by one skilled in the art.

For especially un-gelatinized starch processes the preferred alpha-amylase is an acid alpha-amylase, e.g., fungal acid alpha-amylase or bacterial acid alpha-amylase. The term “acid alpha-amylase” means an alpha-amylase (EC, 3.2.1.1) which added in an effective amount has activity optimum at a pH in the range of 3 to 7, preferably from 3.5 to 6, or more preferably from 4-5.

### Bacterial Alpha-Amylases

Especially for processes including liquefaction of gelatinized starch, alpha-amylases of bacterial origin are preferred.

In a preferred embodiment the alpha-amylase is of Bacillus origin. The Bacillus alpha-amylase may preferably be derived from a strain of B. licheniformis, B. amyloliquefaciens, B. subtilis or B. stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment of the invention the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NO: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. No. 6,093,562, 6,297,038 or 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO: 3 disclosed in WO 99/19467 or deletion of amino acids R179 and G180 using SEQ ID NO: 3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted 1181*+G182*+N193F) compared to the wild-type BSG alpha-amylase amino acid sequence set forth in SEQ ID NO: 3 disclosed in WO 99/19467,

### Bacterial Hybrid Alpha-Amylases

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitution:

G48A+T49I+G107A+H156Y+A181T+N190F+1201F+A209V+Q264S (using the Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and 02648 and/or deletion of two residues between positions 176 and 179, preferably deletion of E178 and G179 (using the SEQ ID NO: 5 numbering of WO 99/19467).

### Fungal Alpha-Amylases

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e., more than 70%, more than 75%, more than 80%, more than 85% more than 90%, more than 95%, more than 96%, more than 97%, more than 98%, more than 99% or even 100% identity to the mature part of the amino acid sequence shown in SEQ ID NO: 10 in WO 96/23874.

Another preferred acidic alpha-amylase is derived from a strain Aspergillus niger. In a preferred embodiment the acid fungal alpha-amylase is the one from A niger disclosed as “AMYA_ASPNG” in the Swiss-prot/TeEMBL database under the primary accession no. P56271 and described in WO 89/01969 (Example 3). A commercially available acid fungal alpha-amylase derived from Aspergillus niger is SP288 (available from Novozymes A/S, Denmark).

Other contemplated wild-type alpha-amylases include those derived from a strain of the genera Rhizomucor and Meriplus, preferably a strain of Rhizomucor pusillus (WO 2004/055178 incorporated by reference) or Meriplus giganteus.

In a preferred embodiment the alpha-amylase is derived from Aspergillus kawachii and disclosed by Kaneko et al., 1996, J. Ferment. Bioeng. 81: 292-298, “Molecular-cloning and determination of the nucleotide-sequence of a gene encoding an acid-stable alpha-amylase from Aspergillus kawachii”; and further as EMBL: # AB008370.

The fungal alpha-amylase may also be a wild-type enzyme comprising a starch-binding domain (SBD) and an alpha-amylase catalytic domain (i.e., non-hybrid), or a variant thereof. In an embodiment the wild-type alpha-amylase is derived from a strain of Aspergillus kawachii.

### Fungal Hybrid Alpha-Amylases

In a preferred embodiment the fungal acid alpha-amylase is a hybrid alpha-amylase. Preferred examples of fungal hybrid alpha-amylases include the ones disclosed in WO 2005/003311 or U.S. Application Publication No. 2005/0054071 (Novozymes) or U.S. Application No. 60/638,614 (Novozymes) which is hereby incorporated by reference, A hybrid alpha-amylase may comprise an alpha-amylase catalytic domain (CD) and a carbohydrate-binding domain/module (CBM), such as a starch binding domain, and optional a linker.

Specific examples of contemplated hybrid alpha-amylases include those disclosed in Table 1 to 5 of the examples in U.S. Application No. 60/638,614, including Fungamyl variant with catalytic domain JA118 and Athelia rolfsii SBD (SEQ ID NO: 100 in U.S. Application No. 60/638,614), Rhizomucor pusillus alpha-amylase with Athelia rolfsii AMG linker and SBD (SEQ ID NO: 101 in U.S. Application No. 60/638,614), Rhizomucor pusillus alpha-amylase with Aspergillus niger glucoamylase linker and SBD (which is disclosed in Table 5 as a combination of amino acid sequences SEQ ID NO: 20, SEQ ID NO: 72 and SEQ ID NO: 96 in U.S. application Ser. No. 11/316,535) or as V039 in Table 5 in WO 2006/069290, and Meripilus giganteus alpha-amylase with Athelia rolfsii glucoamylase linker and SBD (SEQ ID NO: 102 in U.S. Application No, 60/638,614), Other specifically contemplated hybrid alpha-amylases are any of the ones listed in Tables 3, 4, 5, and 6 in Example 4 in U.S. application Ser. No. 11/316,535 and WO 2006/069290 (hereby incorporated by reference).

Other specific examples of contemplated hybrid alpha-amylases include those disclosed in U.S. Application Publication no. 2005/0054071, including those disclosed in Table 3 on page 15, such as Aspergillus niger alpha-amylase with Aspergillus kawachii linker and starch binding domain.

Contemplated are also alpha-amylases which exhibit a high identity to any of above mention alpha-amylases, Le., more than 70%, more than 75%, more than 60%, more than 85%, more than 90%, more than 95%, more than 96%, more than 97%, more than 96%, more than 99% or even 100% identity to the mature enzyme sequences.

An acid alpha-amylases may according to the invention be added in an amount of 0.1 to 10 AFAU/g DS, preferably 0.10 to 5 AFAU/g DS, especially 0.3 to 2 AFAU/g DS,

### Commercial Alpha-Amylase Products

Preferred commercial compositions comprising alpha-amylase include MYCOLASE from DSM, BAN™, TERMAMYL™ SC, FUNGAMYL™, LIQUOZYME™ X and SAN™ SUPER, SAN™ EXTRA L (Novozymes NS) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes A/S, Denmark).

### Carbohydrate-Source Generating Enzymes

The phrase “carbohydrate-source generating enzyme” includes glucoamylase (being glucose generators), beta-amylase and maltogenic amylase (being maltose generators). A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acidic fungal alpha-amylase activity (AFAU) per glucoamylase activity (AGU) (AFAU per AGU) may in an embodiment of the invention be at least 0.1, in particular at least 0.16, such as in the range from 0.12 to 0.50 or more,

### Glucoamylases

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel at al., 1984, EMBO J. 3 (5): 1097-1102), or variants thereof, such as those disclosed in WO 92/00381, WO 00/04136 and WO 01/04273 (from Novozymes, Denmark); the A. awamori glucoamylase disclosed in WO 84/02921, A. oryzae glucoamylase (1991, Agric. Biol. Chem. 55 (4): 941-949), or variants or fragments thereof. Other Aspergillus glucoamylase variants include variants with enhanced thermal stability: G137A and G139A (Chen et al., 1996, Prot. Eng. 9: 499-505); D257E and D293E/Q (Chen et al., 1995, Prot, Eng. 8, 575-582); N182 (Chen et al., 1994, Biochem. J. 301: 275-281); disulphide bonds, A246C (Fierobe et al., 1996, Biochemistry 35: 8698-8704; and introduction of Pro residues in position A435 and 6436 (Li et al., 1997, Protein Eng. 10: 1199-1204.

Other glucoamylases include Athelia rolfsii (previously denoted Corticium rolfsii) glucoamylase (see U.S. Pat. No. 4,727,026 and Nagasaka et al., 1998, “Purification and properties of the raw-starch-degrading glucoamylases from Corticium rolfsii, Appl Microbiol. Biotechnol, 50: 323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. themtchydrosulfuricum (WO 86/01831) and Trametes cingulata disclosed in WO 2006/069289 which is hereby incorporated by reference.

Also hybrid glucoamylase are contemplated according to the invention. Examples the hybrid glucoamylases are, for example, disclosed in WO 2005/045018. Specific examples include the hybrid glucoamylase disclosed in Table 1 and 4 of Example 1 which are hereby incorporated by reference.

Contemplated are also glucoamylases which exhibit a high identity to any of above mention glucoamylases, i.e., more than 70%, more than 75%, more than 80%, more than 85% more than 90%, more than 95%, more than 96%, more than 97%, more than 98%, more than 99% or even 100% identity to the mature enzymes sequences.

Commercially available compositions comprising glucoamylase include AMG 200L; AMG 300L; SAN™ SUPER, SAN™ EXTRA L, SPIRIZYME™ PLUS, SPIRIZYME™ FUEL, SPIRIZYME™ B4U and AMG™ E (from Novozymes NS): OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, especially between 1-5 AGU/g DS, such as 0.5 AGU/g DS.

### Beta-Amylases

At least according to the invention the a beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (Fogarty and Kelly, 1979, Progress in Industrial Microbiology 15: 112-115). These beta-amylases are characterized by having optimum temperatures in the range from 40° C. to 65° C. and optimum pH in the range from 4.5 to 7. A commercially available beta-amylase from barley is NOVOZYM™ WBA from Novozymes NS, Denmark and SPEZYME™ BBA 1500 from Genencor Int., USA.

### Maltogenic Amylases

The amylase may also be a maltogenic alpha-amylase. A maltogenic alpha-amylase (glucan 1.4-alpha-maltohydrolase, E.C. 3.2.1.133) is able to hydrolyze amylose and amylopectin to maltose in the aloha-configuration. A maltogenic amylase from Bacillus stearothermophilus strain NCIB 11837 is commercially available from Novozymes Maltogenic alpha-amylases are described in U.S. Pat. Nos. 4,598,048, 4,604.355 and 6,162,628, which are hereby incorporated by reference.

The maltogenic amylase may in a preferred embodiment be added in an amount of 0.05-5 mg total protein/gram DS or 0.05-5 MANU/g DS.

### Xylose Isomerases

Xylose isomerases (D-xylose ketoisomerase) (E.C. 5.3.1.5.) are enzymes that catalyze the reversible isomerization reaction of D-xylose to D-xylulose. Some xylose isomerases also convert the reversible isomerization of D-glucose to D-fructose. Therefore, xylose isomerase is sometimes referred to as “glucose isomerase”.

A xylose isomerase used in a method or process of the invention may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoplanes, Bacillus and Flavobacterium, and Thermotoga, including T. neapolitana (Vieille et al., 1995, Appl. Environ. Microbial. 61 (5); 1867-1875) and T. maritima.

Examples of fungal xylose isomerases are derived species of Basidiomycetes.

A preferred xylose isomerase is derived from a strain of yeast genus Candida, preferably a strain of Candida boidinii, especially the Candida boidinii xylose isomerase disclosed by, e.g., Vongsuvanlert et al., 1988, Agric. Biol. Chem. 52(7): 1817-1824. The xylose isomerase may preferably be derived from a strain of Candida boidinii (Kloeckera 2201), deposited as DSM 70034 and ATCC 48180, disclosed in Ogata et al., Agric. Biol. Chem. 33: 1519-1520 or Vongsuvanlert at al., 1988, Agric. Biol. Chem. 52(2): 1519-1520.

In one embodiment the xylose isomerase is derived from a strain of Streptomyces, e.g., derived from a strain of Streptomyces marinas (U.S. Pat. No. 4,687,742): S. flavovirens, S. albus, S. achromogenus, S. echinatus, S. wedmorensis all disclosed in U.S. Pat. No. 3,616,221. Other xylose isomerases are disclosed in U.S. Pat. Nos. 3,622,463, 4,351,903, 4,137,126, and 3,625,828, HU patent no. 12,415, DE patent 2,417,642, JP patent no. 69,28,473, and WO 2004/044129, each incorporated by reference herein.

The xylose isomerase may be either in immobilized or liquid form. Liquid form is preferred.

The xylose isomerase is added to provide an activity level in the range from 0.01-100 IGIU per gram total solids.

Examples of commercially available xylose isomerases include SWEETZYME™ T from Novozymes NS, Denmark.

### Proteases

The protease may be any protease. In a preferred embodiment the protease is a acid protease of microbial origin, preferably of fungal or bacterial origin.

Suitable proteases include microbial proteases, such as fungal and bacterial proteases. Preferred proteases are acidic proteases, i.e. proteases characterized by the ability to hydrolyze proteins under acidic conditions below pH 7.

Contemplated acid fungal proteases include fungal proteases derived from Aspergillus, Mucor, Rhizopus, Candida, Coriolus, Endothia, Enthomophtra, Irpex, Penicillium, Sclerotium and Torulopsis. Especially contemplated are proteases derived from Aspergillus niger (see, e.g., Koaze at al., 1964, Agr. Biol. Chem. Japan 28: 216), Aspergillus saltoi (see, e.g., Yoshida, 1954, J. Agr Chem. Soc. Japan 28: 66), Aspergillus awamori (Hayashida et al., 1977, Agric. Biol. Chem. 42(5): 927-933, Aspergillus aculeatus (WO 95/02044), or Aspergillus oryzae, such as the pepA protease: and acidic proteases from Mucor push/us or Mucor miehei.

Contemplated are also neutral or alkaline proteases, such as a protease derived from a strain of Bacillus. A particular protease contemplated for the invention is derived from Bacillus amyloliquefaciens and has the sequence obtainable at Swissprot as Accession No. P06832. Also contemplated are the proteases having at least 90% identity to amino acid sequence obtainable at Swissprot as Accession No. P06832 such as at least 92%, at least 95%, at least 96%, at least 97%, at least 98%, or particularly at least 99% identity. Further contemplated are the proteases having at least 90% identity to amino acid sequence disclosed as SEQ ID NO: 1 in the WO 2003/048353 such as at 92%, at least 95%, at least 96%, at least 97%, at least 98%, or particularly at least 99% identity.

Also contemplated are papain-like proteases such as proteases within E.C. 3.4.22.* (cysteine protease), such as EC 3.4.22.2 (papain), EC 3.4.22.6 (chymopapain), EC 3.4.22.7 (asclepain), EC 3.422.14 (actimidain), EC 3.4, 22.15 (cathepsin L). EC 3.4.22.25 (glycyl endopeptidase) and EC 3.4.22.30 (caricain).

In an embodiment the protease is a protease preparation derived from a strain of Aspergillus, such as Aspergillus oryzae. In another embodiment the protease is derived from a strain of Rhizomucor, preferably Rhizomucor mehei. in another contemplated embodiment the protease is a protease preparation, preferably a mixture of a proteolytic preparation derived from a strain of Aspergillus, such as Aspergillus oryzae, and a protease derived from a strain of Rhizomucor, preferably Rhizomucor meihei.

Aspartic acid proteases are described in, for example, Handbook of Proteolytic Enzymes, Edited by A. J. Barrett, N. D. Rawlings and J. F. Woessner, Academic Press, San Diego, 1998, Chapter 270). Suitable examples of aspartic acid protease include, e.g., those disclosed in Berka et al., 1990, Gene 96: 313): (Berka et al., 1993, Gene 125: 195-198); and Gomi et al., 1993, Biosci. Biotech, Biochem, 57: 1095-1100, which are hereby incorporated by reference.

Commercially available products include ALCALASE®, ESPERASE™, FLAVOURZYME™, PROMIX™, NEUTRASE®, RENNILASE®, NOVOZYM™ FM 2.0L, and NOVOZYM™ 50006 (available from Novozymes A/S, Denmark) and GC106™ and SPEZYME™ FAN from Genencor Int., Inc., USA.

The protease may be present in an amount of 0.0001-1 mg enzyme protein per g DS, preferably 0.001 to 0.1 mg enzyme protein per g DS. Alternatively, the protease may be present in an amount of 0.0001 to 1 LAPU/g DS, preferably 0.001 to 0.1 LAPU/g DS and/or 0.0001 to 1 mAU-RH/g DS, preferably 0.001 to 0.1 mAU-RH/g DS.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties unless otherwise specified. The present invention is further described by the following examples which should not be construed as limiting the scope of the invention.

### Materials & Methods

### Materials

### Enzymes:

Cellulase preparation A: Cellulolytic composition comprising a polypeptide having cellulolytic enhancing activity (GH61A) disclosed in WO 2005/074656; a beta-glucosidase (fusion protein disclosed in U.S. Application No. 60/832,511) and a cellulolytic enzyme preparation derived from Trichoderma reesei. Cellulase preparation A is disclosed in U.S. Application No. 60/941,251.
Glucoamylase SF: Glucoamylase derived from Talaromyces emersonii disclosed as SEQ ID NO: 7 in WO 99/28448 and available from Novozymes NS, Denmark.

### Yeast:

RED STAR™ available from Red Star/Lesaffre, USA

Un-washed acid-treated steam exploded PCS obtained from NREL lot (062706)

Corn mash obtained from HGF Aberdeen, SD, USA.

### Methods

### Determination of Identity

The relatedness between two amino acid sequences or between two nucleotide sequences is described by the parameter “identity,”

The degree of identity between two amino acid sequences may be determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

The degree of identity between two nucleotide sequences may be determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

### Measurement of Cellulase Activity Using Filter Paper Assay (FPU Assay)

### 1. Source of Method

1.1 The method is disclosed in a document entitled “Measurement of Cellulase Activities” by Adney and Baker, 1996, Laboratory Analytical Procedure, LAP-006, National Renewable Energy Laboratory (NREL). It is based on the IUPAC method for measuring cellulase activity (Ghose, 1987, Measurement of Cellulase Activities. Pure & Appl. Chem. 59: 257-268.

### 2. Procedure

2.1 The method is carried out as described by Adney and Baker, 1996, supra, except for the use of a 96 well plates to read the absorbance values after color development, as described below,

### 2.2 Enzyme Assay Tubes:

2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath,
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.

### 2.3 Blank and Controls

2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.

### 2.4 Glucose Standards

2.4.1 A 100 ml., stock solution of glucose (10.0 mg/mL) is prepared, and 5 ml., aliquots are frozen. Prior to use, aliquots are thawed and vortexed to mix.
2.4.2 Dilutions of the stock solution are made in citrate buffer as follows:
   G1=1.0 mL stock+0.5 mL buffer=6.7 mg/mL=3.3 mg/0.5 mL
   G2=0.75 mL stock+0.75 mL buffer=5.0 mg/mL=2.5 mg/0.5 ml.
   G3=0.5 mL stock+1.0 mL buffer=3.3 mg/mL=1.7 mg/0.5 mL
   G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.

### 2.5 Color Development

2.5.1 Following the 60 min. incubation and addition of DNS, the tubes are all boiled together for 5 mins. in a water bath,
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH₂O in a 96-well plate. Each well is mixed, and the absorbance is read at 540 nm.

### 2.6 Calculations (Examples are Given in the NREL Document)

2.6.1 A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.
2.6.2 A plot of glucose produced (mg/0.5 mL) vs. tot enzyme dilution is prepared, with the Y-axis (enzyme dilution) being on a log scale.
2.6.3 A line is drawn between the enzyme dilution that produced just above 2.0 mg glucose and the dilution that produced just below that From this line, it is determined the enzyme dilution that would have produced exactly 2.0 mg of glucose,
2.6.4 The Filter Paper Units/mL (FPU/mL) are calculated as follows:
   FPU/mL=0.37/enzyme dilution producing 2.0 mg glucose

### Glucoamylase Activity

Glucoamylase activity may be measured in AGI units or in Glucoamylase Units (AGU).

### Glucoamylase Activity (AGI)

Glucoamylase (equivalent to amyloglucosidase) converts starch into glucose. The amount of glucose is determined here by the glucose oxidase method for the activity determination. The method described in the section 76-11 Starch-Glucoamylase Method with Subsequent Measurement of Glucose with Glucose Oxidase in “Approved methods of the American Association of Cereal Chemists”. Vol. 1-2 AACC, from American Association of Cereal Chemists, 2000; ISBN: 1-891127-12-8.

One glucoamylase unit (AG) is the quantity of enzyme which will form 1 micro mole of glucose per minute under the standard conditions of the method.

### Standard Conditions/Reaction Conditions:

Substrate: Soluble starch, concentration approx 16 g dry matter/L

Buffer: Acetate, approx. 0.04 M, pH=4.3

pH: 4.3

Incubation temperature: 60° C.

Reaction time: 15 minutes

Termination of the reaction: NaOH to a concentration of approximately 0.2 g/L, (pH˜9)

Enzyme concentration: 0.15-0.55 AAU/mL.

The starch should be Lintner starch, which is a thin-boiling starch used in the laboratory as calorimetric indicator. Lintner starch is obtained by dilute hydrochloric acid treatment of native starch so that it retains the ability to color blue with iodine.

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG incubation:

| | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

Color reaction:

| | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02101) describing this analytical method in more detail is available on request from Novozymes A/S, Denmark, which folder is hereby included by reference.

### Alpha-Amylase Activity

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05, 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Acid Alpha-Amylase Activity

When used according to the present invention the activity of any acid alpha-amylase may be measured in AFAU (Acid Fungal Alpha-amylase Units). Alternatively activity of acid alpha-amylase may be measured in AAU (Acid Alpha-amylase Units).

### Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

### Standard Conditions/Reaction Conditions:

Substrate: Soluble starch. Concentration approx. 20 g DS/L.
Buffer: Citrate, approx. 0.13 M, pH=4.2
Iodine solution: 40.176 g potassium iodide+0.088 g iodine/L
City water 15-20° dH (German degree hardness)
pH: 4.2
Incubation temperature: 30° C.
Reaction time: 11 minutes

### Wavelength: 620 nm

Enzyme concentration: 0.13-0.19 AAU/mL
Enzyme working range: 0.13-0.19 AAU/mL

The starch should be Lintner starch, which is a thin-boiling starch used in the laboratory as calorimetric indicator. Lintner starch is obtained by dilute hydrochloric acid treatment of native starch so that it retains the ability to color blue with iodine, Further details can be found in EP 0140410, which disclosure is hereby included by reference.

### Acid Alpha-Amylase Activity (AFAU)

Acid alpha-amylase activity may be measured in AFAU (Acid Fungal Alpha-amylase Units), which are determined relative to an enzyme standard 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase. E.C. 3.2.1.1) hydrolyzes alpha-1,4-glucosidic bonds in the inner regions of the starch molecule to form dextrins and oligosaccharides with different chain lengths The intensity of color formed with iodine is directly proportional to the concentration of starch. Amylase activity is determined using reverse colorimetry as a reduction in the concentration of starch under the specified analytical conditions.

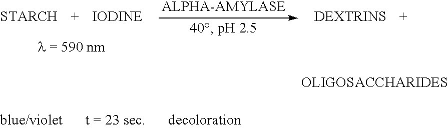

Standard Condition/Reaction Conditions:
Substrate: Soluble starch, approx. 0.17 g/L
Buffer: Citrate, approx. 0.03 M
Iodine (I2): 0.03 g/L
CaCl2: 1.85 mM
pH: 2.50±0.05
Incubation temperature: 40° C.
Reaction time: 23 seconds
Wavelength: 590 nm
Enzyme concentration: 0.025 AFAU/mL
Enzyme working range: 0.01-0.04 AFAU/mL

A folder EB-SM-0259.02/01 describing this analytical method in more detail is available upon request to Novozymes NS, Denmark, which folder is hereby included by reference.

### Xylose/Glucose Isomerase Assay (IGIU)

1 IGIU is the amount of enzyme which converts glucose to fructose at an initial rate of 1 micromole per minute at standard analytical conditions,

Standard Conditions.

Glucose concentration: 45% w/w

pH: 7.5

Temperature: 60° C.

Mg²⁺ concentration: 99 mg/l (1.0 g/l MgSO₄*7H₂O)

Ca²⁺ concentration<2 ppm

Activator, SO₂ concentration: 100 ppm (0.18 g/l NaS₂O₅)

Buffer, Na₂CO₃, concentration: 2 mM

### Protease Activity

### Protease Assay Method (LAPU)

1 Leucine Amino Peptidase Unit (LAPU) is the amount of enzyme which decomposes 1 microM substrate per minute at the following conditions: 26 mM of L-leucine-p-nitroanilide as substrate, 0.1 M Tris buffer (pH 8.0), 37° C., 10 minutes reaction time.

LAPU is described in EB-SM-0298.02/01 available from Novozymes NS Denmark on request.

### Protease Assay Method—AU(RH)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TCA soluble product is determined with phenol reagent, which gives a blue color with tyrosine and tryptophan.

One Anson Unit (AU(RH)) is defined as the amount of enzyme which under standard conditions (i.e., 25° C., pH 5.5 and 10 min. reaction time) digests hemoglobin at an initial rate such that there is liberated per minute an amount of TCA soluble product which gives the same color with phenol reagent as one milliequivalent of tyrosine.

AU(RH) is described in EAL-SM-0350 available from Novozymes A/S Denmark on request.

### Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for minutes.

### EXAMPLES

### Example 1

The purpose of this experiment is to study the effect of introducing pre-treated lignocellulose-containing material (in this case PCS filtrate) into a starch-containing material based fermentation process (in this case corn mash fermentation (SSF) process).

### PCS Filtrate

Un-washed acid-treated steam exploded PCS (i.e., pre-treated corn stover) was hydrolyzed for 48 hours at 15% TS (total solids) in a batch reactor at pH 5 (PCS adjusted using 1 M NH₄OH) and 50° C. Cellulase preparation A was dosed at 5 mg-EP/g-TS (appr. 15 mg-EP/g-CEL). After hydrolysis the PCS hydrolyzate was filtered in a Buchner funnel under vacuum using glassfiber filterpaper (Whatman: GF/D) and finally the filtrate was sterile-filtered. The PCS filtrate (PCS-t) was analyzed using HPLC (see Table 1 1). The hydrolysis yield represents a cellulose conversion (glucose only) of 90%.

TABLE 1
Composition of PCS filtrate. All concentrations are in g/L

| Cellobiose | Glucose | Xylose | Arabinose | Glycerol | HAc | Ethanol |
|---|---|---|---|---|---|---|
| 2.3 | 48.3 | 29.1 | 3.9 | 0.2 | 5.3 | 0.0 |

A lab scale SSF process was run with corn mash at 31.7% DS (from corn mash only), in which PCS filtrate was added at different volumes according to scheme below (Table 22). Glucoamylase SF (825 AGU/g) was dosed at 0.45 AGU/g-DS (corn mash (CM) only) and SSF was run at 32(C. RED STAR™ yeast was re-hydrated at 32° C. for 30 minutes before inoculation with a cell concentration in each batch at about 1.2 g/L. Five tubes of each CM/PCS-f combination was run in 15 mL snap-cap tubes with hole dulled in top with a reference containing only CM (no PSC-f added). Weight loss was determined over the course of SSF for 68 hours using HPLC.

TABLE 2
Ratios of corn mash (CM) and PCS tested for integrated SSF

| Sample | PCS filtrate ratio |
|---|---|
| 1 | 0 ml/5 g-CM |
| 2 | 0.5 ml/5 g-CM |
| 3 | 1.0 ml/5 g-CM |
| 4 | 1.5 ml/5 g-CM |
| 5 | 2.0 ml/5 g-CM |
| 6 | 2.5 ml/5 g-CM |
| 7 | 3.0 ml/5 g-CM |

### HPLC Analysis

Concentrations of cellobiose, glucose, xylose, arabinose, glycerol, acetic acid (HAc) and ethanol were determined using HLPC analysis (Agilent HP-1100 system) on a Biorad HPX-87H organic acid column with R1-detection. All the above compounds were quantified using calibrated standards.

### Results

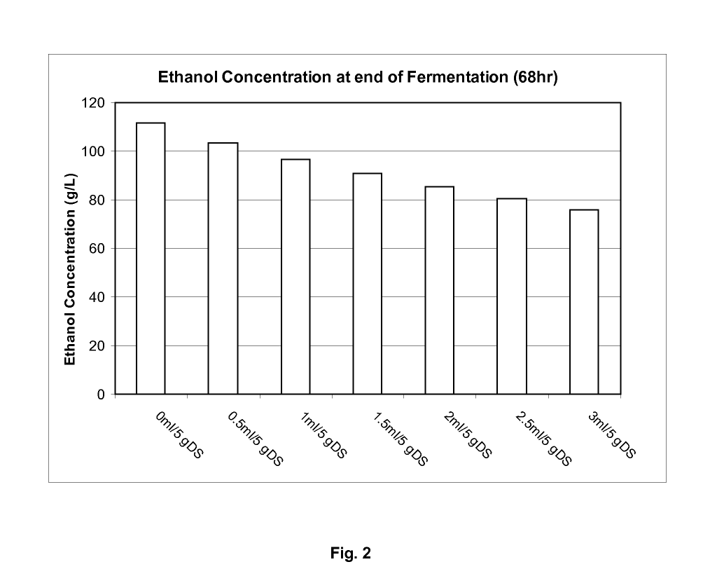

The results of the integrated fermentation is presented in FIG. 1 as ethanol yield per corn mash solids (g-EtOH/g-DS), which was based on weight loss data collected frequently during fermentation. FIG. 2 shows the HPLC data at the end of fermentation. The weight loss data does not take into account the added glucose from PCS-f and that the HPLC data does not take into account the additional volume added with the PCS. With both of these facts taken into account, assuming a stoichiometric conversion of the glucose form the PCS-f to ethanol during fermentation, the fermentation yields (at end of fermentation) were found to be at the same level (Table 3). These results indicate that introduction of PCS do not have negative impact on conventional corn mash fermentation.

TABLE 3
Ethanol concentrations by the end of fermentation (68 hours).

| | | Ethanol | Relative to |
|---|---|---|---|
| PCS liquid ratio | Sample | (g/L) | Reference |
| 0 ml/5 gDS | 1 | 111.5 | 100% |
| 0.5 ml/5 gDS | 2 | 111.3 | 100% |
| 1 ml/5 gDS | 3 | 111.8 | 100% |
| 1.5 ml/5 gDS | 4 | 112.2 | 101% |
| 2 ml/5 gDS | 5 | 112.2 | 101% |
| 2.5 ml/5 gDS | 6 | 112.2 | 101% |
| 3 ml/5 gDS | 7 | 112.4 | 101% |

Concentrations corrected for dilution and ethanol potential from PCS glucose.
Reference: corn mash SSF without any PCS-f

1. A method for producing a fermentation product from lignocellulose-containing material, wherein the method comprises:
i) pre-treating lignocellulose-containing material;
ii) introducing pre-treated lignocellulose-containing material into a medium comprising fermentable sugars derived from starch-containing material; and
ii) fermenting using a fermenting organism.

2. The method of claim 1, wherein the starch-containing material and the lignocellulose-containing material are treated in two separate streams before saccharification, fermentation or simultaneous saccharification and fermentation.

3. The method of claim 1, wherein the medium is a starch saccharification medium, starch fermentation medium or starch simultaneous saccharification and fermentation medium.

4. The method of claim 1, wherein the method comprises introducing pre-treated lignocellulose-containing material into a simultaneous saccharification and fermentation medium containing one or more starch-degrading enzymes and optionally a fermenting organism.

5. The method of claim 1, wherein the fermentation is initiated before or after the pre-treated lignocellulose material is introduced into the medium.

6. The method of claim 1, wherein the pre-treated lignocellulose-containing material is hydrolyzed before fermentation or simultaneous saccharification and fermentation.

7. The method of claim 1, wherein solids from the pre-treated lignocellulose-containing material are removed before or during fermentation.

8. The method of claim 1, wherein the pre-treated lignocellulose-containing material, having solids removed, is added during saccharification, fermentation, or simultaneous saccharification and fermentation.

9. The method of claim 1, wherein the pre-treated lignocellulose-containing material is un-detoxified.

10. The method of claim 1, wherein the lignocellulose-containing material has been chemically, mechanical or biologically pre-treated.

11. The method of claim 1, wherein the lignocellulose-containing material has been hydrolyzed by treatment with one or more cellulases or hemicellulases, or a combination thereof.

12. The method of claim 1, wherein the starch-containing material is liquefied gelatinized starch-containing material.

13. The method of claim 1, wherein the liquefied gelatinized starch-containing material is saccharified before or during fermentation.

14. The method of claim 1, wherein the starch-containing material is uncooked starch-containing material.

15. The method of claim 1, wherein the lignocellulose derived material introduced into the fermentation medium is un-washed.

16. A process for producing a fermentation product from a combination of starch-containing material and lignocellulose-containing material comprising the steps of:
a) liquefying starch-containing material;
b) saccharifying;
c) fermenting using a fermenting organism;
wherein the pre-treated lignocellulose-containing material is added before or during fermentation.

17. The process of claim 16, wherein saccharification in step (b) and fermentation in step (c) are carried out sequentially or simultaneously.

18. The method of claim 16, wherein the pre-treated lignocellulose-containing material is hydrolyzed before fermentation or simultaneous saccharification and fermentation.

19. The method of claim 16, wherein the starch-containing material and the lignocellulose-containing material are treated in two separate streams before saccharification, fermentation or simultaneous saccharification and fermentation.

20. The process of claim 16, wherein solids from the pre-treated lignocellulose-containing material are removed before fermentation.

21. The process of claim 16, wherein the pre-treated lignocellulose-containing material, having solids removed, is added to saccharification step b), fermentation step c), or simultaneous saccharification and fermentation.

22. The method of claim 21, wherein the pre-treated lignocellulose-containing material is un-detoxified.

23. The process of claim 16, wherein the lignocellulose-containing material has been chemically, mechanically or biologically pre-treated.

24. A process for producing a fermentation product from a combination of starch-containing material and lignocellulose-containing material comprising the steps of:
i) saccharifying the starch-containing material at a temperature below the initial gelatinization temperature;
ii) fermenting using a fermenting organism;
wherein the pre-treated lignocellulose containing material is added before or during fermentation.

25. The process of claim 24, wherein saccharification in step i) and fermentation in step ii) are carried out sequentially or simultaneously.

26. The process of claim 24, wherein the pre-treated lignocellulose-containing material is hydrolyzed before fermentation or simultaneous saccharification and fermentation.

27. The process of claim 24, wherein the starch-containing material and the lignocellulose-containing material are treated in two separate streams before saccharification, fermentation or simultaneous saccharification and fermentation.

28. The process of claim 24, wherein the pre-treated lignocellulose material has further been hydrolyzed by treatment with a cellulase or hemicellulase, or a combination thereof, before fermentation.

29. The process of claim 24, wherein solids from the pre-treated lignocellulose-containing material are removed before fermentation.

30. The process of claim 24, wherein the pre-treated lignocellulose-containing material, having solids removed, are added to saccharification step i), fermentation step ii) or simultaneous saccharification and fermentation.

31. The process of claim 24, wherein the lignocellulose-containing material has been chemically, mechanically or biologically pre-treated.

32. The process of claim 24, wherein one or more carbohydrate-generating enzymes are used during saccharification or simultaneous saccharification and fermentation.

33. The process of claim 24, wherein the lignocellulose-containing material is un-washed.

34. The process of claim 24, wherein the starch-containing material is uncooked granular starch.
